(19) United States
(12) Patent Application Publication
(10) Pub. No.: US 2005/0033210 A1
(43) Pub. Date: Feb. 10, 2005

(54) Soft Contact Patch for Treatment of Amblyopia

(76) Inventor: Parsa Shahinpoor, Albuquerque, NM (US)

Correspondence Address: MOHSEN SHAHINPOOR, 909 VIRGINIA, NE, SUITE 205, ALBERQUERQUE, NM, 87108, US

(21) Appl. No.: 10/604,655

(22) Filed: Aug. 7, 2003

(51) Int. Cl. A61F 13/00

(52) U.S. Cl. 602/41

(57) ABSTRACT

Specially designed soft contact patch for treating amblyopia (lazy eye) in children and possibly adults is disclosed. A soft contact patch designed such that its pupil section is opaque and essentially black or tinted dark and its iris section surrounding the pupil section is large enough to snugly fit the eye under the eyelid and is either transparent or colored to show or match the natural color of the eye, and it is perforated and porous to allow oxygenating the iris and the sclera, so that for selected portions of time the stronger eye may be occluded. Thus, the stronger eye can be occluded for a desirable period of time to force the weaker amblyopic eye to work harder and become stronger in time to correct amblyopia.

### BACKGROUND OF INVENTION

1. Field of the Invention

This invention relates to the treatment of amblyopia, also known as “lazy eye”, in children and possibly adults for whom the resolving power of one eye (strong eye) is more than the resolving power of another eye (weak eye or lazy eye).

2. Background of the Invention

Amblyopia or commonly referred to as Lazy Eye is a common visual problem afflicting approximately 4% of the population with about some 200,000 new cases occurring yearly in the United States. Amblyopia (lazy eye) is probably the most common cause of monocular blindness.

It occurs when the vision pathways in the brain fail to develop normally, resulting in impaired vision. According to the NIH National Eye Institute in the US, Amblyopia (“Lazy Eye”) is the leading cause of vision loss in one eye in people aged 20-70+, surpassing diabetes, glaucoma and cataract.

The most common causes of Amblyopia, which develops during childhood, are strabismus (crossed eyes) or anisometropia (one eye being strongly near- or far-sighted compared to the other eye). In normal eyesight, the brain receives images from each eye and merges them into one. In Amblyopia, the brain receives strong images or neural input from one eye and weak images from the other (lazy-eye) and thus the brain ignores images from the weaker—lazy eye. As a result, the brain's vision system for that eye fails to develop normally. This neurological condition is believed to occur due to a neural input imbalance of either the optical power of the eyes or ocular misalignment. Both of these conditions may result in an incompatible binocular visual input to the visual centers of the brain that prevents a normal, single visual perception. This incompatibility of visual perception induces a competitive inhibition between the two eyes resulting in a “strong eye” and a “weak eye”. Here, the visual utility of the “strong eye” becomes dominant over the “weak eye”. This results in permanent structural degradation of the cellular anatomy of the eye and visual cortex. By definition, an amblyopic eye has a visual acuity worse than 20/30. Visual acuity in an amblyopic eye can range from 20/30 to 20/200 (clinically blind) and worse. Because people with lazy eye primarily rely on vision from one eye, they lack stereovision (three-dimensional viewing ability). Their depth perception is impaired, and peripheral and night visions can also be weakened. After visual maturity of the child is reached, which is about 10 years of age, the disease becomes permanent. Over time, the lazy eye becomes weak and loss of vision occurs. At this stage, the problem may not be corrected with glasses, surgery or other optical measures, since the problem is related to the brain and brain optical pathways and not to the eye.

Lazy Eye is correctable if detected and treated early (before the age of 8 or 9). Early detection is a key factor in successful treatment by a pediatric ophthalmologist. The accepted treatment of amblyopia involves blocking or reducing vision in the strong eye, either by a patch or eye drop (atropine) to blur the vision of the good eye or specially-tinted glasses in order to force the weaker eye to “work harder”. This establishes and reinforces the development of neural pathways in the brain to cause proper connections to develop between the weak, amblyopic eye and the visual cortex. After a period of time, which may be between months and years, use of the patch is gradually reduced, affording both eyes the opportunity to develop normal binocular vision. Atropine and patching produce improvement of similar magnitude, and both are appropriate modalities for the initial treatment of moderate amblyopia in children aged 3 to less than 7 years.

Patching or covering one eye may be required for a period of time ranging from a few weeks to as long as a year. Medication in the form of eye drops or ointment may be used to blur the vision of the good eye in order to force the weaker one to work. This is generally a less successful approach.

Patching, where a child must wear an eye patch over the stronger eye for an extended period of time, is the most common form of treatment. However, many children do not comply with their treatment schedule because patches may be viewed as uncomfortable or socially embarrassing. Since the stronger eye is patched, children must learn to cope with poorer vision in the weaker eye until it becomes stronger. For these reasons, combined with the fact that amblyopia often goes undetected in childhood, many children become adults with amblyopia.

### SUMMARY OF INVENTION

The present invention is directed towards specially designed soft contact patch for treating diseases of the class including amblyopia (lazy eye) in children and possibly adults. The said invention in the form of soft contact patch is such that its pupil section is opaque and essentially black or tinted dark and its iris section surrounding the pupil section is large enough to snugly fit the inside of the eye under the eyelid like a soft contact lens and is either transparent or colored to show or match the natural color of the eye or the iris, and it is perforated and porous to allow oxygenating the iris and the sclera, so that for selected portions of time the stronger eye may be occluded or partially occluded (tinted pupil). Thus, the stronger eye can be occluded or partially occluded for a desirable period of time to force the weaker amblyopic eye to work harder and become stronger in time to correct amblyopia.

### BRIEF DESCRIPTION OF DRAWINGS

FIG. 1 depicts an embodiment of the present invention in the form of specially designed opaque soft contact patch.

FIG. 2 depicts another embodiment of the present invention wherein the soft contact patch is shown in the eye.

### DETAILED DESCRIPTION

The present invention is directed at using a specially designed soft contact patch with an opaque or tinted pupil section and either transparent or colored iris section for improving visual acuity of an individual in which the loss of acuity is the result of a “lazy eye” or amblyopia. The condition known as amblyopia or lazy eye can be overcome by exercising the eye according to the method of the invention. Specially designed soft contact patch for treating amblyopia (lazy eye) in children and possibly adults is disclosed. A soft contact patch designed such that its pupil section is opaque and essentially black or tinted dark and its iris section surrounding the pupil section is large enough to snugly fit inside the eye under the eyelid like a soft contact lens and is either transparent or colored to show or match the natural color of the eye or the iris, and it is perforated and porous to allow oxygenating the iris and the sclera, so that for selected portions of time the stronger eye may be occluded or partially occluded. Thus, the stronger eye can be occluded or partially occluded for a desirable period of time to force the weaker amblyopic eye to work harder and become stronger in time to correct amblyopia.

Because there are several causes of amblyopia, the treatment must match the problem. Glasses fix some problems. Surgery may be needed for cataracts, droopy eyelids (ptosis) or crossed eyes (strabismus). After the cause of the amblyopia is found, say in a child, the child with amblyopic eye will need to use the weaker eye most of the time, so it will get stronger. To make the child use the weaker eye, a patch can be put over the stronger eye. Sometimes, eye drops or special glasses are used to blur the vision in the stronger eye. This makes the weaker eye become stronger. Patches may be used all-day or part of the day, depending on the child's age and vision. The treatment usually lasts until vision is normal, or until vision stops getting better. For most children, this takes several weeks. A few children need to use eye patches until they are 8 to 10 years old.

The vision pathways in the brain must become strong early, when children are very young. The first few years of life are the most important for eyesight. After a child is 8 to 10, the brain's vision system is complete. It can't develop anymore. If the amblyopia hasn't been treated by this age, the child will have poor vision for life. It won't be possible to fix it with glasses, patching or any other treatment.

There's a small chance that using an eye patch for too long can hurt the strong eye. For this reason, children who are wearing eye patches should see their doctor often during the treatment.

Eye patches have been discussed in a number of patents. For example, in U.S. Pat. No. 4,867,146, issued Sep. 19, 1989 and entitled Eye patch, Krupnick, et al., discuss an eye patch for preventing opening of an eye and preventing corneal abrasion. The patch comprises a compressible planar pad having a configuration, which fits, within the eye socket to permit easy contact with the outer surface of the eyelids over a closed eye. The planar pad is flexible to facilitate conforming to the outer surface of the lids of the eye. One surface of the pad has an adhesive with a low to moderate adhesion applied thereto to permit securement of the lids in a closed position while permitting removal of the patch with minimal patient discomfort. In U.S. Pat. No. 4,951,658, issued Aug. 28, 1990 and entitled Eye patch with hydrocolliod adhesive, Morgan, et al., discuss an eye patch having a backing member with an inner periphery having a hydrocolloidal adhesive for securing the patch over a person's eye and an inner fabric material for contacting the eyelid. C. Gail Summers (MD) and James E. Egbert (MD) have reported in American Orthoptic journal (Am Orthopt j46:111-117, 1996) in 1996 regarding a case study of occluder contact lens tolerance in noncompliant patients with amblyopia. In their study amblyopic children who were noncompliant with an occlusive patch were provided with an occluder black contact lens as an alternative for amblyopia therapy. In U.S. Pat. No. D446, 307 issued Aug. 7, 2001 and entitled Eye Patch, Bassett presents an ornamental design for an eye patch, as shown and described. In U.S. Pat. No. 6,320,094, issued Nov. 20, 2001 and entitled Disposable eye patch and method of manufacturing a disposable eye patch, Arnold, et al., discusses disposable eye patch includes a first sheet member, a second sheet member smaller than the first sheet member, and an adhesive layer applied to one side of the first sheet member. One portion of the adhesive layer bonds the first and second sheet members together, and another portion of the adhesive layer adheres to the tissue surrounding an eye when the eye patch is applied to a patient. A release layer may be provided to cover the exposed portion of the adhesive layer, and may be peeled off prior to the use of the eye patch. The release layer and a plurality of eye patches may be provided in the form of a dispenser roll assembly. In a method of manufacturing the disposable eye patch, the first and second sheet members may both be formed from a sheet material having an adhesive layer, and then the first and second sheet members may be adhered together with the adhesive layer on one of the sheet members contacting the adhesive layer on the other one of the sheet members. New types of patches are still being developed and used. See for example the recent patent by Ashton, et. al, entitled Eye patch for treatment of amblyopia, U.S. Pat. No. D466,610 issued Dec. 2, 2002, that presents new ornamental design for an eye patch for treatment of amblyopia, as shown and described.

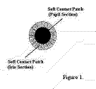

It turns out that no patent has been issued for an eye patch in the form of a specially designed soft contact patch with an opaque or essentially black or tinted pupil section, as shown in FIG. 1, and either transparent or colored iris section for improving visual acuity of an individual in which the loss of acuity is the result of a “lazy eye” or amblyopia. The condition known as amblyopia or lazy eye can be overcome by exercising the eye according to the method of the invention. Therefore, an object of the present invention is to provide a new means for correcting amblyopia, which will be more attractive to traditional treatment using either an external eye patch or eye drop or tinted glasses.

In accordance with one embodiment of this invention, and referring to the basic example shown in FIG. 1, a soft contact patch is manufactured to substantially cover the iris and the pupil of the strong eye of amblyopic individual such that its pupil section is completely opaque or tinted with a large aperture while its iris section is either transparent to show the real color of the eye or the iris or colored to match the color of the eye or the iris or a different color. Furthermore, the iris section of the soft patch is circular like a soft contact lens and is rather geometrically compatible with the geometry of the eye to snugly fit the inside of the eye under the eyelid and is further perforated or porous so that oxygen can reach the iris and the sclera.

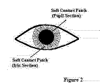

FIG. 2 depicts the amblyopic soft contact patch in the strong eye of a user further showing that its presence in the eye will look fairly natural and will eliminate the problem with wearing a patch over the eye, which may be socially embarrassing. Also note the soft contact patch pupil section and the iris section in FIG. 2 covering the iris section and pupil section of the eye.

Since it will be difficult for a child to place the soft contact patch in his or her strong eye, the parents should learn how to place a soft contact patch in a child's strong eye and educate their child to observe the basic rules of wearing a soft contact and to also remove the soft contact from the child's eye when desired.

This invention has been described with reference to specific embodiments. Those of ordinary skill in the art will understand that variations in these methods and compositions may be used and that it is intended that the invention may be practiced otherwise than as specifically described herein. Accordingly, this invention includes all modifications encompassed within the spirit and scope of the invention as defined by the claims.

1. A specially designed soft contact patch for treating eye disorders of the class including amblyopia or lazy eye in children and possibly adults such that:
The pupil section is opaque and essentially black or tinted dark and its iris section surrounding the pupil section is large enough to snugly fit inside the eye under the eyelid and is either transparent or colored to show or match the natural color of the eye, and it is perforated and porous to allow oxygenation of the iris and the sclera, so that for selected portions of time the stronger eye may be occluded or partially occluded.

2. An invention as described in claim 1 wherein said specially designed soft contact patch is used in the occlusion of the stronger eye for selected portions of time, thus, the stronger eye can be occluded for a desirable period of time to force the weaker amblyopic eye to work harder and become stronger in time to correct amblyopia.

3. A system as set forth in claims 1 and 2 wherein said specially designed soft contact patch is equipped with and displays graphical designs.

4. A system as set forth in claim 1 wherein said specially designed soft contact patch is equipped with and displays a single or a variety of colors.

5. An invention as described in claims 1, 2 and 3, wherein a pair of soft contact patches are used such that the one used in the strong eye is the soft contact patch to block or partially block the vision by means of a dark or tinted pupil section and the other soft contact is used like a soft contact lens to enable the weak eye correct its own refractive errors.

6. The soft contact patch of claim 1, 2, 3 and 4 wherein said soft contact patch comprises polymethyl methacrylate.

7. The soft contact patch of claim 1, 2, 3 and 4 wherein said soft contact patch comprises silicone.

8. The soft contact patch of claim 1, 2, 3 and 4 wherein said soft contact patch comprises a combination of polymethyl methacrylate and silicone.

9. The soft contact patch of claim 1, 2, 3 and 4 wherein said soft contact patch comprises cellulose acetate butyrate.

10. The soft contact patch of claim 1, 2, 3 and 4 wherein said soft contact patch comprises a gas permeable material.

11. The soft contact patch of claim 1, 2, 3 and 4 wherein said soft contact patch comprises polyhydroxyethyl-methacrylate (HEMA), copolymers of HEMA and N-vinylpyrrolidone (NVP), silicone or other material known to be useful in the fabrication of soft contact lenses.

12. The soft contact patch of claim 1, 2,3 and 4 wherein said soft contact patch comprises tinted pupil section made with typical polysilicone-acrylate polymer containing a typical photochromic colorant.

13. The soft contact patch of claim 1, 2,3 and 4 wherein said soft contact patch comprises conventional soft polymeric gel materials used in conventional soft contact lenses.
